(19) United States
(12) Patent Application Publication
(10) Pub. No.: US 2026/0257036 A1
(43) Pub. Date: Sep. 3, 2026

(54) URINARY CATHETER WITH OVERMOLDED FUNNEL

(71) Applicant: Hollister Incorporated, Libertyville, IL (US)

(72) Inventors: Daniel A. March, Lake Villa, IL (US); Jon Lugenbill, Warrenville, IL (US); Jeremy Quint, Round Lake Beach, IL (US); Scott J. Pupino, Lakewood, IL (US)

(21) Appl. No.: 18/878,750

(22) PCT Filed: Jun. 27, 2023
(86) PCT No.: PCT/US2023/069193

§ 371 Date: Dec. 24, 2024

Related U.S. Application Data
(60) Provisional application No. 63/356,372, filed on Jun. 28, 2022.

(51) Int. Cl. A61M 25/00

(52) U.S. Cl. CPC A61M 25/0017; A61M 25/0009

(57) ABSTRACT

A urinary catheter includes a funnel body having a first body section and a second body section extending from the first body section. The first body section has a first mass and the second body section has a second mass less than the first mass. The funnel body is made from TPE and a funnel passage extends through the funnel body. The catheter also includes a catheter tube having a first tube segment, a transition segment and a second tube segment. The funnel body is overmolded on the first tube segment, the transition segment extends outward from the second body section, and the second segment extends outward from the transition segment. The transition segment has a smaller tube wall thickness than at least one of the first tube segment and the second tube segment.

The present application claims the benefit of and priority to U.S. Provisional Application No. 63/356,372, filed Jun. 28, 2022, which is hereby incorporated by reference.

### DESCRIPTION

### TECHNICAL FIELD

The present disclosure generally relates to urinary catheters, a method and an apparatus for manufacturing the urinary catheters, and more particularly, to urinary catheters having a funnel overmolded on a catheter tube.

### BACKGROUND

Urinary catheters include a tube and a funnel. It is desirable maintain physical integrity during use of the catheters, for example, by providing a sufficiently strong bond between the tube and the funnel. It is also desirable for catheters to facilitate easy insertion and removable from the user's urethra.

To this end, the funnel of a known urinary catheter may be made from Polyvinyl chloride (PVC) and include a gripping portion on an outer surface to facilitate easy handling of the catheter. PVC has been selected due to material characteristics which satisfy various user and/or industry preferences. For example, PVC has been found to have material characteristics that are beneficial in manufacturing, for example, in injection molding processes.

However, use and disposal of PVC is becoming more regulated. Accordingly, there is a need to provide a urinary catheter, method and apparatus for manufacturing a urinary catheter using alternative materials.

### SUMMARY

In one aspect, a urinary catheter includes a funnel body having a first body section and a second body section extending from the first body section. The first body section has a first mass and the second body section has a second mass less than the first mass. A distal end of the funnel body is on the first body section and a proximal end of the funnel body is on the second body section. The funnel body is made from TPE and a funnel passage extends through the funnel body. The urinary catheter also includes a catheter tube having a first tube segment, a transition segment and a second tube segment. The funnel body is overmolded on the first tube segment, the transition segment extends outward from the second body section, and the second segment extends outward from the transition segment. The transition segment has a smaller tube wall thickness than at least one of the first tube segment and the second tube segment.

In another aspect, a method of manufacturing a urinary catheter having a funnel body and a catheter tube includes positioning the catheter tube in a molding apparatus, the molding apparatus having a molding plate assembly formed with a funnel body mold cavity and a shutoff plate assembly formed with a shutoff cavity, the catheter tube extending in the shutoff cavity and into the funnel body mold cavity. The method also includes positioning a core pin in the funnel body mold cavity and inserting the core pin into the catheter tube and preloading a portion of the catheter tube in the shutoff plate assembly. The method further includes feeding moldable material into the funnel body mold cavity and a funnel part of the shutoff cavity at a selected temperature and pressure. Feeding moldable the material includes feeding a first mass of the moldable material into the funnel body molding cavity and a second mass of the moldable material into the funnel part, the second mass being less than the first mass and configured to cool and solidify before the first mass. The first mass of the moldable material forms a first body section of the funnel body and the second mass of the moldable material forms a second body section of the funnel body. The method also includes removing the core pin to form a funnel passage in the funnel body.

In another aspect, a molding apparatus for manufacturing a urinary catheter having a funnel body and a catheter tube includes a molding plate assembly having a funnel body mold cavity and a core pin arranged in the funnel body mold cavity, the funnel body mold cavity configured to receive a first mass of a moldable material. The molding apparatus further includes a shutoff plate assembly adjacent to the molding plate assembly. The shutoff plate assembly includes a shutoff cavity having a funnel part and a tube part, the funnel part configured to receive a second mass of the moldable material, less than the first mass, the tube part configured for preloading a portion of the catheter tube positioned in the tube part.

### BRIEF DESCRIPTION OF THE FIGURES

FIG. 1 is cross-sectional view of urinary catheter according to examples of the present disclosure;

FIGS. 2A and 2B are cross-sectional views showing examples of funnel bodies of urinary catheters according to the present disclosure;

FIG. 3 is an enlarged view of a proximal end of a funnel body according to the present disclosure;

FIG. 4 is a cross-sectional view of a catheter tube according to examples of the present disclosure;

FIG. 5 is a schematic illustration of a molding apparatus for manufacturing a urinary catheter according to the present disclosure;

FIG. 6 is an enlarged view of a shutoff plate assembly of the molding apparatus of FIG. 5;

FIG. 7 is an enlarged view a shutoff passage of the shutoff plate assembly of FIG. 6;

FIG. 8 is a side view of a core pin of the molding apparatus of FIG. 5;

FIG. 9 is a cross-sectional view of the core pin and funnel body molded around the core pin;

FIG. 10 is a side view showing the urinary catheter in the molding apparats before removal of the core pin; and

FIG. 11 is a block diagram showing a method for manufacturing a urinary catheter according to present embodiments.

### DETAILED DESCRIPTION OF THE ILLUSTRATED EMBODIMENTS

The embodiments disclosed herein are for the purpose of providing a description of the present subject matter, and it is understood that the subject matter may be embodied in various other forms and combinations not shown in detail. Therefore, specific embodiments and features disclosed herein are not to be interpreted as limiting the subject matter as defined in the accompanying claims.

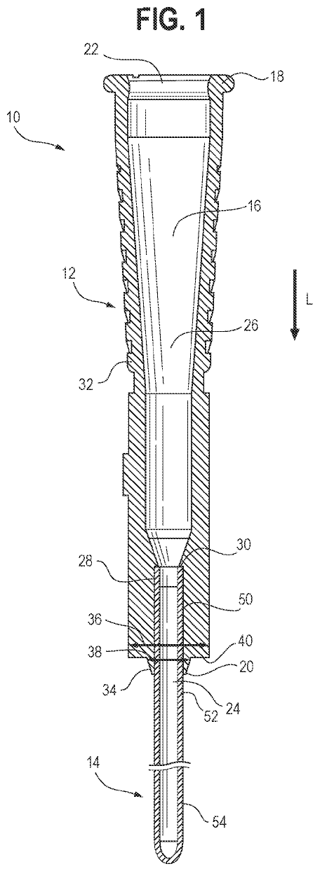

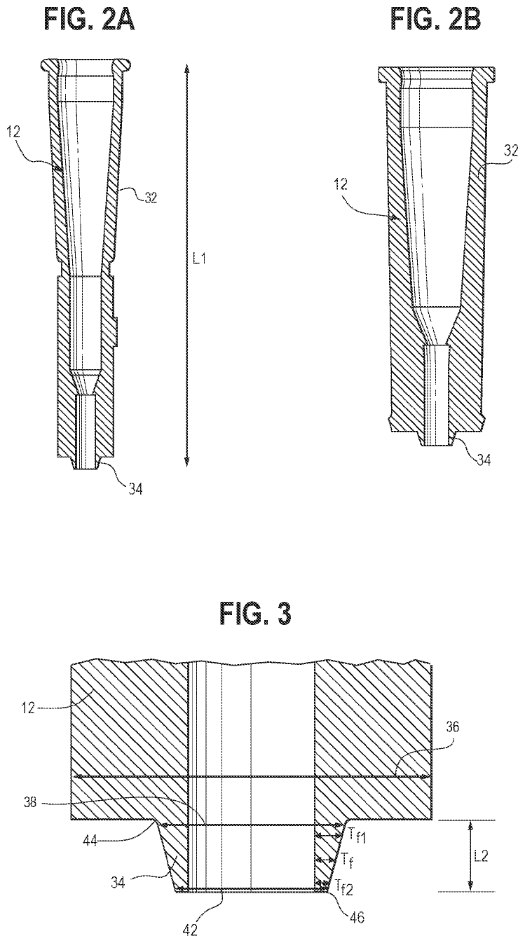

Referring to FIGS. 1, 2A and 2B, a urinary catheter 10 according to the present embodiments generally includes a funnel body 12 and a catheter tube coupled to the funnel body 12. The funnel body 12 is coupled the catheter tube 14 by overmolding the funnel body 12 on the catheter tube 14. In one example, the funnel body 12 is made from a moldable material, such as a thermoplastic elastomer (TPE) material. The catheter tube 14 may also be made from a TPE material.

A funnel passage 16 extends through the funnel body 12 and is surrounded by an inner surface of the funnel body 12. Thus, a diameter of the funnel passage 16 corresponds to an inner diameter of the funnel body 12. The funnel passage 16 extends in a length direction L of the funnel body 12 from a distal end 18 to a proximal end 20 of the funnel body 12. A first opening 22 of the funnel passage 16 is arranged at the distal end 18 and a second opening 24 of the funnel passage 16 is arranged at the proximal end 20.

The funnel passage 16 includes a funnel section 26 and an insert section 28 arranged in series in the length direction L of the funnel body 12. The funnel section 26 includes one or more segments of decreasing diameter in the length direction L from the distal end 18 to the proximal end 20. The insert section 28 surrounds and is bonded to a portion of the catheter tube 14. The inner surface may include an inner shoulder 30 extending in a radial direction and facing in the length direction L toward the proximal end 20.

The funnel body 12 includes a first body section 32 and a second body section 34 extending from the first body section 32 in the length direction L. The distal end 18 is arranged on the first body section 32 and the proximal end 20 is arranged on the second body section 34. In one example, funnel section 26 of the funnel passage 16 extends through the first body section 32 of the funnel body 12 and the insert section 28 extends in the first body section 32 and the second body section 34 of the funnel body 12. The first body section 32 and the second body section 34 are continuous with another and formed as a single piece.

The first body section 32 has a first outer diameter 36 at an end opposite to the distal end 18, generally where the first body section 32 is connected to the second body section 34. The second body section 34 has a second outer diameter 38 at an end opposite to the proximal end 20, generally where the second body section 34 is connected to the first body section 32. The first outer diameter 36 is larger than the second outer diameter 38 such that a radial step or outer shoulder 40 is formed where the first body section 32 is connected to the second body section 34. The outer shoulder 40 extends from the first outer diameter 36 to the second outer diameter 38 and faces in the length direction L toward the proximal end 20. A plane defined by the outer shoulder 40 may generally be considered as a boundary between the first body section 32 and the second body section 34.

The funnel body 12 is formed from a moldable material, e.g., TPE.

The first body section 32 has a first mass of the moldable material and the second body section 34 has a second mass of the moldable material. The second mass is less than the first mass. The second body section 34 is sized and shaped so that the second mass of material, in a molding process, cools and solidifies before the first mass of material. In this manner, the material of the second body section 34 bonds to the catheter tube 14 and may substantially seal against or otherwise resist leakage of moldable material from a proximal end of the molding apparatus during the molding process.

In addition, the second mass of material allows for relatively quick cooling and solidifying of the material during the molding process. Thus, exposure time of the catheter tube 14 arranged in the second body section 34 to heat and pressure of the molding process may be limited. Accordingly, deformation and/or melting of the catheter tube 14 as a result of exposure to heat and/or pressure may be limited. Limiting deformation and/or melting of the catheter tube 14 in the second body section 34 may promote sealing the proximal end of the molding apparatus against leakage of the moldable material under heat and pressure during the molding process.

Referring to FIG. 3, the second body section 34 may further include a third outer diameter 42 at the proximal end 20. The third outer diameter 42 is less than the second outer diameter 38 such that an outer surface of the second body section 34 has a slope or curvature. In one example, the second body section may be substantially conical or frustoconical in shape.

As indicated above, the second body section 34 is sized and shaped to promote cooling and solidifying of the moldable material before cooling and solidifying the moldable material of the first body section 32. To this end, various geometric parameters are considered in manufacturing, e.g., in selecting or manufacturing the molding apparatus, to achieve the desired cooling and solidification effect with respect to time. Examples of such geometric parameters of the second body section 34 may include a length L2 and a wall thickness T_{f}. In some examples, the wall thickness may vary in the length direction L2.

In some examples, the length L2 of the second body section 34 may be between 1.3 mm and 1.7 mm. In another example, the length L2 of the second body section 34 may be between 1.4 mm and 1.6 mm. In one example, the length L2 of the second body section 34 is about 1.5 mm. The length L2 of the second body section 34 is less than the length L1 of the first body section 32.

In some examples, the wall thickness T_{f} may be between 0.13 mm and 0.86 mm. In another example, the wall thickness T_{f} may be between 0.14 mm and 0.85 mm. In one example, the wall thickness T_{f} may be between about 0.15 mm and 0.84 mm.

In some examples, the wall thickness T_{f} varies along the length L2 of the second body section 34. For example, the second body section 34 may have a first (or maximum) wall thickness T_{f1} and a second (or minimum) wall thickness T_{f2} between which the wall thickness T_{f} varies along the length L2. In some examples, the first wall thickness T_{f1} may be provided adjacent to the first body section 32 and the second wall thickness T_{f2} may be provided, for example, at the proximal end 20. In some examples, the wall thickness T_{f} may decrease gradually along the length L2 of second body section 34. In the illustrated examples, the wall thickness T_{f} decreases linearly along the length L2 of the second body section 34. However, it will be appreciated that the present disclosure is not limited to such a geometry. For example, the wall thickness T_{f} may decrease is such a manner to form a curved outer surface (e.g., according to an exponential formula, arc, oval, and the like) In further examples, the second body section 34 may include various combinations of cylindrical (e.g., constant wall thickness), sloped (linear decreasing wall thickness toward proximal end), stepped, and/or curved portions.

In some examples, the first or maximum wall thickness T_{f1} may between about 0.82 mm and 0.86 mm. In further examples, the first or maximum wall thickness T_{f1} may be between about 0.83 mm and 0.85 mm. In one example, the first or maximum wall thickness T_{f1} may be about 0.84 mm.

In some examples, the second or minimum wall thickness T_{f2} may be between about 0.13 mm and 0.17 mm. In further examples, the second or minimum wall thickness T_{f2} may be between about 0.14 mm and 0.16 mm. In one example, the second or minimum wall thickness T_{f2} may be about 0.15 mm.

It will be appreciated that various combinations of the above examples may be used together.

In some examples, the outer surface of the second body section 34 may extend at a non-right angle from the outer shoulder 40 of the first body section 32. For example, the second body section 34 may be angled between about 100 degrees and 110 degrees, and in a further example, between about 104 degrees and 105 degrees.

In some examples, the second body section 34 may be formed having a first curvature 44 adjacent to the first body section 32. The first curvature 44 may have a radius between about 0.008 inches and 0.012 inches (i.e., about 0.203 mm to 0.305 mm), and in a further example, may be about 0.010 inches (i.e., about 0.254 mm). Alternatively, or in addition, the second body section 34 may be formed having a second curvature 46 at the proximal end 20. The second curvature 46 may be between about 0.003 inches and 0.007 inches (i.e., about 0.076 mm to 0.178 mm), and in a further example, may be about 0.005 inches (i.e., about 0.127 mm).

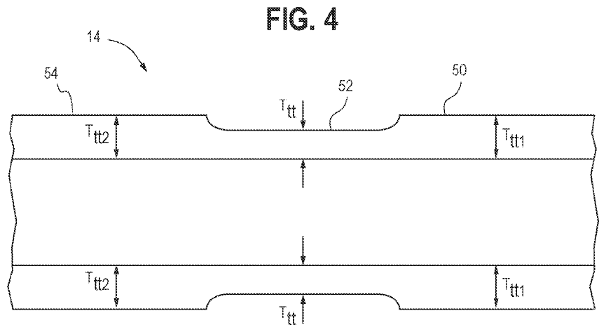

Referring to FIG. 4, the catheter tube 14 includes a first tube segment 50 arranged in the insert section 28 of the funnel body 12, a transition segment 52 adjacent to and extending away from the second body section 34, and a second tube segment 54 extending from the transition segment 52. Thus, the transition segment 52 may be arranged between the first tube segment 50 and the second tube segment 54.

The transition segment 52 may be compressed relative to the first tube segment 50 and the second tube segment 54. The transition segment 52 may be compressed, for example, during the molding process of the funnel body 12. In one example, a portion of the catheter tube 14 is preloaded in the molding apparatus, and the transition segment 52 is formed along the preloaded portion.

The transition segment 52 may have a reduced tube wall thickness Tₜₜ relative to the wall thickness Tₜₜ₁ of first tube segment 50 and the wall thickness Tₜₜ₂ second tube segment 54. In addition, the transition segment 52 may have a higher resistance to bending and kinking relative to the second tube segment 54. The catheter tube 14 may include a first radius section transitioning from the transition segment 52 wall thickness Tₜₜ to the first tube segment 50 wall thickness Tₜₜ₁. The catheter tube 14 may include a second radius section transitioning from the transition segment 52 wall thickness Tₜₜ to the second tube segment 54 wall thickness Tₜₜ₂. Although the example illustrated in FIG. 4 shows the reduced tube wall thickness Tₜₜ at the transition segment 52 formed having a reduced outer diameter relative to the outer diameters of the first and/or second tube segments 50, 54, it will be appreciated that the reduced tube wall thickness Tₜₜ may alternatively, or additionally, be formed having an increased inner diameter relative to the inner diameters of the first and/or second tube segments 50, 54.

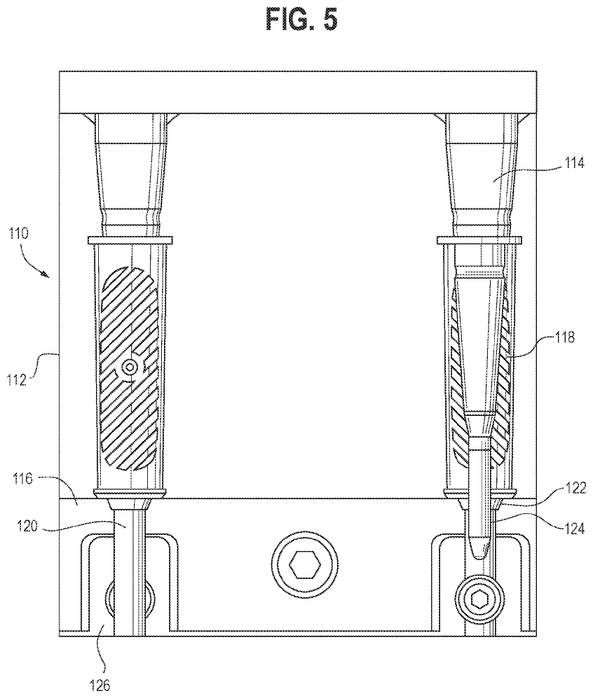

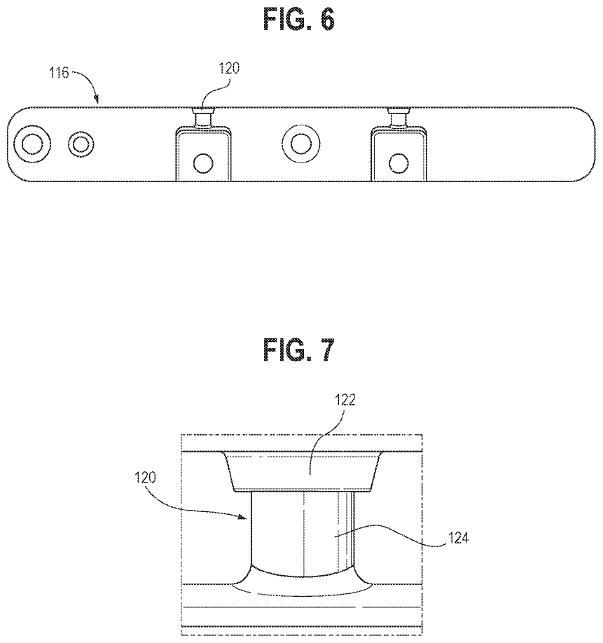

Referring to FIGS. 5-8, a molding apparatus 110 for manufacturing the urinary catheter 10 according to present embodiment includes, for example, a molding plate assembly 112, a core pin 114 and a shutoff plate assembly 116.

The molding plate assembly 112 includes a funnel body mold cavity 118 and the core pin 114 may extend through the funnel body mold cavity 118 during the molding process. Accordingly, in the molding process, moldable material may be supplied to the funnel body mold cavity 118 at a selected temperature and pressure. The moldable material in the funnel body mold cavity 118 surrounds the core pin 114. Upon cooling and solidifying, the moldable material in the funnel body mold cavity 118 forms that first body section 32 of the funnel body 12. The core pin 114 is sized and shaped to correspond to the size and shape of the funnel passage 16. Accordingly, the core pin 114 is removed from the cooled material leaving an internal void which forms the funnel passage 16.

The shutoff plate assembly 116 is arranged adjacent to the molding plate assembly 112. The shutoff plate assembly 116 includes a shutoff cavity 120 having a funnel cavity part 122 and a tube part 124. The shutoff cavity 120 is aligned with the funnel body mold cavity 118 such that a portion of the core pin 114 may be received in the shutoff cavity 120.

In an example of the molding process, the moldable material fed into the funnel body mold cavity 118 flows into the funnel cavity part 122 of the shutoff cavity 120. The moldable material cools in the funnel cavity part 122 to form the second body section 34 of the funnel body 12 integral with the first body section 32.

The catheter tube 14 is positioned partially within the funnel body mold cavity 118 and extends through the shutoff cavity 120. In the molding process, the core pin 114 is inserted into a portion of the catheter tube 14 and extends at least partially though the shutoff cavity 120. The moldable material in the funnel body mold cavity 118 and the funnel cavity part 122 of the shutoff cavity 120 is overmolded on the portions of the catheter tube 14 arranged in the funnel body mold cavity 120 and the shutoff cavity 122. The funnel body 12 is bonded to the catheter tube 14 in this manner.

A preload is applied to a portion of the catheter tube 14 arranged in the tube part 124 of the shutoff plate assembly 116 to compress the catheter tube 14, thereby forming the transition segment 52. The preload includes an external preload applied to an external surface of the catheter tube 14 from the shutoff plate assembly 116. In one example, the shutoff plate assembly 116 is formed, at least in part, by opposing shutoff plates which are brought into contact with one another to apply the external preload to the catheter tube 14. The preload may also include an internal preload applied to an internal surface of the catheter tube 14 from the core pin 114. The core pin 114 may extend into the portion of the catheter tube 14 arranged in the tube part 124. With the shutoff plates 116 in contact with one another, the walls of catheter tube 14 may be pressed between the shutoff plates 116 on the external surface and the core pin 114 on the internal surface. Accordingly, the shutoff plates 116 may apply the external preload and the core pin 114 may apply the internal preload, resulting in final preload or compression on the portion of the catheter tube 14 arranged in the tube part 124 to form the transition segment 52. In various examples, an inner diameter of the tube part 124 is less than an outer diameter of the uncompressed catheter tube 14. Thus, the external preload may be applied by bringing the shutoff plates 116 into contact with one another around the catheter tube 14. Additionally, in some examples, the core pin 114 may have a diameter that is equal to or greater than an inner diameter of the catheter tube 14. Thus, the internal preload may be applied by the core pin 114 when the shutoff plates 116 are brought into contact with one another around the catheter tube 14 which, in turn, may cause the core pin 114 to apply reaction force as the internal preload.

In various examples, the catheter tube 14 may be compressed by about 0.06 mm to 0.20 mm per side, and in further examples, by about 0.08 mm to 0.18 mm per side. Further, in some examples, the catheter tube 14 may be provided in different tube sizes (e.g., different inner/outer diameters), and compression of the catheter tube 14 may vary depending on the catheter tube size. For example, for one or more catheter tube sizes, the catheter tube 14 may be compressed by about 0.06 mm to 0.11 mm per side, and in further examples, between about 0.08 mm and 0.09 mm per side. For another one or more catheter tube sizes, the catheter tube 14 may be compressed by about 0.16 mm to about 0.20 mm per side, and in further examples by about 0.18 mm per side. The compressed portions of the catheter tube 14 may result in a reduced wall thickness relative to the first tube segment 50 and second tube segment 54.

The applied preload further serves to tightly press the shutoff plates against the portion of the catheter tube 14 arranged in the shutoff plates. The applied pressure, in addition to compressing the catheter tube, provides a sealing force around the catheter tube 14 to resist leakage of the moldable material through the shutoff plate assembly 116 during the molding operation.

The catheter 10 of the present embodiments may be formed by injection molding the funnel body 12 on to a preformed catheter tube 14. That is, the funnel body 12 may be formed as an overmold on the catheter tube 14. In some embodiments, the funnel body 12 is made from TPE. The catheter tube 14 may be made from TPE as well.

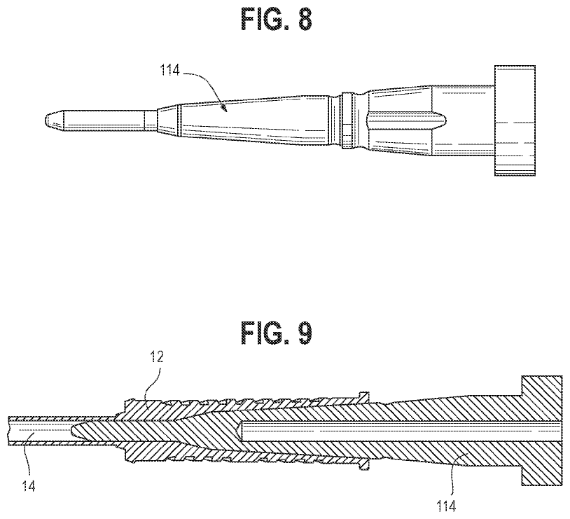

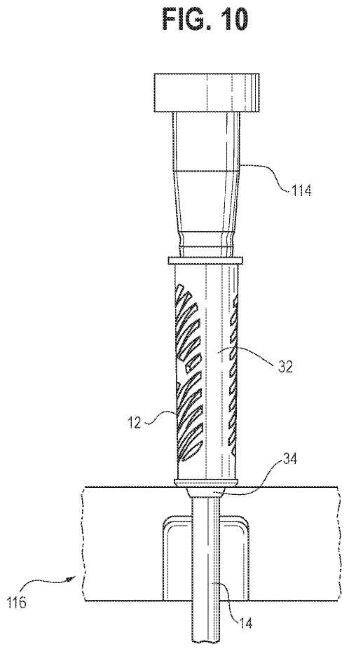

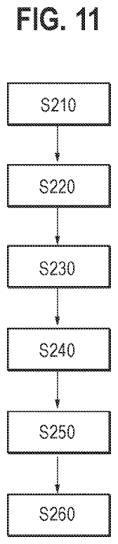

Referring to FIGS. 8-11, a method of manufacturing the urinary catheter 10 according to present embodiments includes, at S210, positioning the catheter tube 14 in the molding apparatus 110. Positioning the catheter 14 may include positioning the catheter tube 14 in a tube holder 126 and positioning the tube holder 126 relative to the molding apparatus 110 to arrange a portion of the catheter tube in the shutoff plate assembly 116 and the molding plate assembly 112. For example, a portion of the catheter tube 14 may be positioned in the shutoff cavity 120 and the funnel body mold cavity 118 (see, e.g. FIGS. 5 and 10).

At S220, the core pin 114 is positioned in the funnel body mold cavity 118 and is inserted into the catheter tube 14 (see, e.g., FIG. 9).

At S230, the method includes preloading a portion of the catheter tube 14 in the shutoff plate assembly 116. Preloading the portion of the catheter tube 14 in the shutoff plate assembly 116 includes preloading the portion of the catheter tube 14 positioned in the tube part 124. Preloading the portion of the catheter tube 14 also compresses the portion of the catheter tube 14 and may reduce a wall thickness of the catheter tube 14. Preloading the catheter tube 14 also serves to resist leakage of the moldable material through the shutoff plate assembly 116 during the molding process.

At S240, the method includes feeding the moldable material into the funnel body mold cavity 118 and the funnel part 122 of the shutoff cavity 120 at a selected temperature and pressure for injection molding. The funnel body mold cavity 118 is configured to receive a first mass of the moldable material and the funnel part 122 is configured to receive a second mass of the moldable material. The second mass is less than the first mass and is configured to cool and solidify before the first mass in the funnel body mold cavity 118. In this manner, the moldable material in the funnel part 122 may bond to the catheter tube 14 and resist leakage of the moldable material through the funnel part 122.

Once the moldable material is cooled and solidified in the molding apparatus 110, at S250, the core pin 114 is removed. The remaining moldable material in the funnel body mold cavity 118 and the funnel part 122 form the funnel body 112 of the urinary catheter. The funnel body 12 is overmolded on the first tube segment 50 and transition segment 52 extends outward from the second body section 34 of the funnel body 12. At S260, the urinary catheter 10 may be removed from the molding apparatus 110.

It will be understood that the embodiments described above are illustrative of some of the applications of the principles of the present subject matter. Numerous modifications may be made by those skilled in the art without departing from the spirit and scope of the claimed subject matter, including those combinations of features that are individually disclosed or claimed herein. For these reasons, the scope hereof is not limited to the above description but is as set forth in the following claims, and it is understood that claims may be directed to the features hereof, including as combinations of features that are individually disclosed or claimed herein.

What is claimed is:

1. A urinary catheter comprising:
a funnel body having a first body section and a second body section extending from the first body section in a length direction of the funnel body, wherein the first body section has a first mass and the second body section has a second mass less than the first mass, wherein a distal end of the funnel body is on the first body section and a proximal end of the funnel body is on the second body section, and wherein the funnel body is made from TPE;
a funnel passage extending through the funnel body in the length direction; and
a catheter tube having a first tube segment, a transition segment and a second tube segment;
wherein the funnel body is overmolded on the first tube segment, the transition segment extends outward from the second body section, and the second segment extends outward from the transition segment; and
wherein the transition segment has a smaller tube wall thickness than at least one of the first tube segment and the second tube segment.

2. The urinary catheter of claim 1, wherein the funnel passage has a funnel section and an insert section and the first tube segment is positioned in the insert section.

3. The urinary catheter of claim 1, wherein the funnel passage has a first opening at the distal end and a second opening at the proximal end.

4. The urinary catheter of claim 1, wherein the first body section has a first outer diameter and the second body section has a second outer diameter, wherein the second outer diameter is less than the first outer diameter.

5. The urinary catheter of claim 4, wherein the second outer diameter decreases along the length direction toward the proximal end.

6. The urinary catheter of claim 5, wherein the second body section is conical or frustoconical in shape.

7. The urinary catheter of claim 1, wherein the first body section has a first length and the second body section and a second length, wherein the second length is less than the first length.

8. The urinary catheter of claim 7, wherein the second length is between 1.3 mm and 1.7 mm.

9. The urinary catheter of claim 8, wherein the second length is about 1.5 mm.

10. The urinary catheter of claim 1, wherein the second body section has a section wall thickness and the section wall thickness decreases along the length direction toward the proximal end.

11. The urinary catheter of claim 10, wherein the section wall thickness includes a first wall thickness adjacent to the first body section and a second wall thickness at the proximal end.

12. The urinary catheter of claim 11, wherein the first wall thickness is between about 0.82 mm to 0.86 mm.

13. The urinary catheter of claim 12, wherein the first wall thickness is about 0.84 mm.

14. The urinary catheter of claim 11, wherein the second wall thickness is between about 0.13 mm to 0.17 mm.

15. The urinary catheter of claim 14, wherein the second wall thickness is about 0.15 mm.

16. A method of manufacturing a urinary catheter having a funnel body and a catheter tube, the method comprising:
positioning the catheter tube in a molding apparatus, the molding apparatus having a molding plate assembly formed with a funnel body molding cavity and a shutoff plate assembly formed with a shutoff cavity, the catheter tube extending in the shutoff cavity and into the funnel body molding cavity;
positioning a core pin in the funnel body mold cavity and inserting the core pin into the catheter tube;
preloading a portion of the catheter tube in the shutoff plate assembly;
feeding moldable material into the funnel body mold cavity and a funnel part of the shutoff cavity at a selected temperature and pressure, wherein feeding moldable material includes feeding a first mass of the moldable material into the funnel body molding cavity and a second mass of the moldable material into the funnel part, the second mass being less than the first mass and configured to cool and solidify before the first mass, and wherein the first mass of the moldable material forms a first body section of the funnel body and the second mass of the moldable material forms a second body section of the funnel body; and
removing the core pin to form a funnel passage in the funnel body.

17. The method of claim 16, wherein the second body section is conical or frustoconical in shape.

18. The method of claim 16, wherein the second body section of the funnel body has a length between 1.3 mm and 1.7 mm.

19. The method of claim 16, wherein the second body section has a first wall thickness adjacent to the first body section and a second wall thickness at a proximal end, wherein the first wall thickness is between about 0.82 mm and 0.86 mm and the second wall thickness is between about 0.13 mm and 0.17 mm.

20. A molding apparatus for manufacturing a urinary catheter having a funnel body and a catheter tube, the molding apparatus comprising:
a molding plate assembly having a funnel body mold cavity and a core pin arranged in the funnel body mold cavity, the funnel body mold cavity configured to receive a first mass of a moldable material; and
a shutoff plate assembly adjacent to the molding plate assembly, the shutoff plate assembly having a shutoff cavity including a funnel part and a tube part, the funnel part configured to receive a second mass of the moldable material, less than the first mass, the tube part configured for preloading a portion of the catheter tube positioned in the tube part.
